(19) United States
(12) Patent Application Publication
(10) Pub. No.: US 2026/0283865 A1
(43) Pub. Date: Sep. 24, 2026

(54) FLUSHABLE ABSORBENT PAD AND COMBINATION WITH REUSABLE CHASSIS

(71) Applicant: W. PELZ GMBH & CO KG, Wahlstedt (DE)

(72) Inventors: Henning RÖTTGER, Kaltenkirchen (DE); Matthias WALLERT, Klein Ronnau (DE)

(73) Assignee: W. PELZ GMBH & CO KG, Wahlstedt (DE)

(21) Appl. No.: 19/474,572

(22) PCT Filed: Mar. 27, 2024
(86) PCT No.: PCT/EP2024/058231

§ 371 Date: Oct. 10, 2025

(30) Foreign Application Priority Data
Apr. 14, 2023 (EP) 23167957.2

(51) Int. Cl. A61F 13/505; A61F 13/15; A61F 13/42; A61F 13/534; A61L 15/28

(52) U.S. Cl. CPC A61F 13/505; A61F 13/42; A61L 15/28; A61F 2013/15357; A61F 2013/15463; A61F 2013/5349

(57) ABSTRACT

The present invention is an absorbent pad suitable to be flushed, such as in a toilet and especially adapted to be fitted into a chassis, such that a hygiene article system is formed.

### FIELD OF THE INVENTION

The present invention relates to an absorbent pad suitable to be flushed, such as in a toilet and especially adapted to be fitted into a chassis, such that a hygiene article system is formed.

### BACKGROUND

Apart from washable cloth diapers, two-piece diaper systems comprising a reusable and washable outer layer and a disposable insert have long been used. Over the last decades, disposable diapers have significantly improved both ease of application and liquid handling performance, which further led to healthier skin of a wearer even at extended wear times, not only for babies but also for incontinent adults as well as for menstruating women. Especially for the latter two categories, absorbent pads are broadly used for lower liquid handling requirements, such as light to medium incontinence or lower menstrual flow conditions, whereby such pads are typically inserted into regular underwear, optionally attached thereto by releasable adhesive, or—mostly in hospitals or care facilities—elasticated pants. In particular for mobile people, discreetness during wear times and ease of replacement become highly relevant. However, with an increased environmental awareness, disposable products have come under scrutiny for using non-renewable resources and/or for an increase carbon footprint in the overall lifecycle, including treatment of the used products. Also, the disposability of hygiene articles, such as wipes, has been broadly discussed and for flushability stringent requirements have been developed.

U.S. Pat. No. 3,489,149 (Larson) describes a re-usable and washable panty for menstrual wear having in the crotch portion a pocket for an optional absorbent insert or pad, wherein the user-oriented portion of the pocket is formed from a first hydrophobic, yet moisture-passing layer for improved skin dryness oriented towards the wearer and a hydrophilic layer oriented towards the insert, if present, or the panty material, which has some tendency to retain moisture for the case when not used in combination with an insert pad. The insert may be disposable.

In EP2376046, a wearable absorbent article to be worn by a wearer about the lower torso is described, comprising a re-useable chassis adapted to receive an absorbent insert therein. The chassis comprises insert fastener components and the absorbent insert comprises a pair of elasticized standing cuffs.

EP0549988 (K-C) describes a disposable article, such as a diaper, for absorbing and containing urine and other body exudates having an absorbent insert pad that may be flushed in a toilet and which may be positioned within a pocket. A cover is releasably attached to a bodyside liner to maintain the insert pad within the pocket. The insert pad includes an absorbent material, which may include wood pulp fluff and high-absorbency materials, such as well-known superabsorbent materials. The absorbent material may be wrapped in a flushable, dispersible carrier sheet, as may comprise a low-wet-strength cellulosic tissue or a nonwoven material such as a rayon carded web that is bonded with polyvinyl alcohol. The ends of the carrier sheet may be folded onto themselves and bonded using sonic bonds, adhesives, such as time sensitive binder such as a polyvinyl alcohol-based adhesive. The longitudinally− extending sides of the carrier sheet may overlap slightly and are preferably unbonded to allow absorbent material to escape from within the carrier sheet once the insert pad has been deposited in a toilet

EP0549988 (K-C) describes a disposable article with an absorbent insert pad (21) that may be flushed in a toilet. The absorbent insert may comprise fibrous material, such as fluff pulp and particulate material, such as superabsorbent polymer particles, which may be wrapped in a flushable, dispersible carrier sheet, such as cellulose tissue. The carrier sheet may be wrapping the absorbent material in “C-type” overfolding, whereby the longitudinal overlap region may remain unbonded and the front and rear ends are bonded using sonic bonds, adhesives, such as a polyvinyl alcohol-based adhesive, which functions as a time sensitive binder that will break down rapidly after being deposited in toilet bowl water.

U.S. Pat. No. 6,623,466 describes an absorbent article with a releasably attached absorbent pouch, which are bonded thermally or adhesively. The absorbent pouch may comprise flushable and degradable materials.

US20170266064 describes a unitary absorbent article for which the components, especially the absorbent core, may be separated from each other and disposed separately, whereby the absorbent core may be flushable, biodegradable and compostable. Optionally, the absorbent material may be wrapped by a membrane. The bonding of all elements, including core and the wrapping membrane is achieved by conventional methods and materials, such by adhesives, heat bonds, pressure bonds, ultrasonic bonds, mechanical bonds, or embossing.

U.S. Pat. No. 5,207,662 describes a two-piece chassis-absorbent-pad system, wherein the absorbent material of the pad is wrapped in a low strength low adhesive non-woven casing.

Co-pending application WO2023/180584 (Pelz) describes a system with a re-usable and washable article chassis that is adapted to receive an absorbent core, so as to ease replacing of the core upon loading, addressing improved positioning but also exchange of a loaded absorbent core. As described, the absorbent core may be contained by an upper and a lower cover sheet, which are connected to each other by means like adhesive or thermobonding.

However, in course of recent developments, further criteria have been developed to address flushability of nonwoven materials, see e.g., EDANA flushability requirements for materials and wipes, EDANA GD.4 and further tests as detailed in the Test Methods section herein below. For a product to be deemed flushable there must be evidence indicating that it quickly disintegrates and does not clog toilets and properly maintained drainage pipe systems, passes through wastewater conveyance systems and is compatible with wastewater treatment, reuse and disposal systems without causing system blockage, clogging or other operational problems; and is unrecognizable in effluent leaving on-site and municipal wastewater treatment systems and in digested sludge from wastewater treatment plants that are applied to soil, essentially satisfying certain bio-degradability criteria.

Whilst such criteria have been developed with a focus on flushable wipes, their principles also apply to all potentially flushable nonwoven and/or hygiene products. When adopting the flushability criteria to hygiene articles as a combination of such materials all satisfying these criteria, a first challenge remains of not introducing structural elements that induce non-disintegration of the article. Such elements can be any water-insensitive connecting, but also the above-described water-soluble glues may lose adhesive properties in excess of water too slowly. A further challenge for designing flushable absorbent products relates to longer wearing period of hygiene articles compared to the wipe products for which the criteria have been developed, in particular as during the prolonged wear period wetting of the article results in change of properties, especially reduction of mechanical strength respectively integrity of the article or its elements during use.

Thus, the present invention gives a solution by providing a hygiene article system comprising a quickly disintegrating flushable absorbent pad as may be combined with a particular hygiene chassis comprising a pocket adapted to receive such a pad.

### SUMMARY

In a first aspect, the resent invention is a flushable and degradable absorbent pad comprising an absorbent core and a wrap material and exhibiting a length (x-), width (y−), and thickness (z−) direction and comprising a front, a rear, and two lateral portions.

The absorbent core comprises absorbent material, preferably comprising fibrous and particulate material. The wrap material is overfolded along fold lines, and covers all x-y− directionally extending surfaces of the absorbent core by at least one layer thereof. Further the fold lines encircle the absorbent core at least laterally and rearwardly, and optionally leave a front opening section un-overfolded. When the contour of the absorbent core and the fold lines are projected onto an x-y− directional plane, all vector lines directing away at 90° from the contour of the absorbent core are crossing a projection of a fold line, except if they cross a projection of the front opening section, if present, which is positioned width-wise extending tangentially at the furthermost front point of the core contour between laterally outermost longitudinally extending tangents to the contour.

Preferably, absorbent core and the wrap material are consisting essentially of materials satisfying EDANA GD.4 flushability test requirements, adapted to the materials as described herein, and wherein the absorbent core satisfies one or more requirements selected from the group consisting of
a) flushability criteria as described herein;
b) industrial composting according to DIN EN 13432;
c) garden composting according to NF T51-800:2015;
d) disintegrating faster than the wrap web in the slosh box test included in the GD.4 requirements.

The absorbent pad may further satisfy one or more of the elements selected from the group consisting of
e) the absorbent pad being essentially free of adhesive or thermoplastic bonding;
f) the absorbent pad being essentially unbonded or by mechanically induced hydrogen bonding only;
g) the absorbent pad exhibiting an absorbent centrifuge capacity of
   g1) more than about 500 ml/m², preferably more than about 1000 ml/m², more preferably more than about 2000 ml/m², and most preferably of more than about 3000 ml/m²; or
   g2) less than 30000 ml/m², preferably less than about 20000 ml/m²;
h) the absorbent pad comprises absorbent particles based on modified starch or carboxymethyl cellulose;
i) the absorbent pad comprises cellulose based fibers;
j) the absorbent core comprises wetness sensitive embossing pattern.

An absorbent pad may even further comprise one or more of the elements selected from the group consisting of
k) the wrapping is essentially unbonded or by mechanically bonding induced hydrogen bonding and/or hydroentangling only;
l) the wrapping being essentially free of adhesive or thermoplastic bonding;
m) the wrapping comprises overfold lines of the wrap web that are completely circumscribing the absorbent core;
n) the wrap web comprises a wetness sensitive embossing pattern.

In another aspect, the present invention is an absorbent system comprising such an absorbent pad and further comprises a re-usable chassis comprising a pocket adapted to receiving the pad.

Preferably, in such an absorbent system the absorbent pad does not disintegrate upon submitting the system with an absorbent pad inserted into the pocket to the slosh box test as modified to apply to an article.

Further, the absorbent core of such an absorbent system is exhibiting along the length direction of the absorbent pad a dry stiffness of less than 20°, preferably less than 15°, more preferably less than about 10°, according to the Cantilever test, as described herein, and a wet stiffness at 50% of its design capacity, as defined herein, of less than about 12 cm, preferably less than about 10 cm, more preferably less than about 9 cm, each to reach a bending angle of 41.5° according to the Cantilever test, as described herein.

### BRIEF DESCRIPTION OF THE FIGURES

FIG. 1A and B depict principles of a two-piece system with an absorbent pad to be positioned in a chassis as known from prior art.

FIGS. 1C and D depict a two-piece system with an absorbent pad as may be according to the present invention.

FIG. 2A to D And FIG. 3 depict executions of an absorbent pad as known from prior art.

FIG. 4A to G depict a particular execution of an absorbent pad according to the present invention.

FIGS. 5 and 6 show preferred features according to the present invention.

FIG. 7A to D depict an absorbent pad according to the present invention.

FIG. 8A to D depict further particular executions of absorbent cores suitable for the present invention.

FIGS. 9A and B depict a particular execution of an absorbent pad according to the present invention.

FIG. 10 A to C depict alternative overfolding options for overfolding a wrap material in an absorbent pad according to the present invention.

The figures are schematically only, and not to scale. Same numerals refer to same or equivalent features or elements, single (′) or multiple (“, ′”, . . . ) apostrophes indicate multiple features, such a left and right or front and back, etc.

### DETAILED DESCRIPTION

In a first aspect, the present invention is an absorbent pad, which is suitable for being flushed in a toilet and exhibits a moderate absorbency;
immediate disintegration;
complete biodegradability;
and good manageability for a user, both in a dry and in a wet state.

Thus, an absorbent pad should at least satisfy the flushability criteria as established for flushable wipes, as summarized in the EDANA specification GD.4 or equivalent, adapted for be useful for complete articles. Preferably, such an absorbent pad should also be suitable for being disposed via the composting route, be this by satisfying the requirements for industrial composting according to DIN EN 13432 or equivalent, or even for home or garden composting, according to NF T51-800:2015, or equivalent, or any further regulation relating to compositing in combination with biodegradability as listed in the Methods Section herein below. Also, requirements for chemical and biological oxygen demand should be satisfied, such as according to biodegradability testing and VDI 4630 criteria for waste water sludge treatment, or other regulations as listed in the Methods Section.

However, functional liquid absorbency during use and rapid disintegration are design contradictions. Thus, in a first aspect, the present invention is an absorbent pad, which in itself is not suitable for use, except when being combined with a fixation aid, also referred to as a chassis, that ensures functional integrity of the absorbent pad during wear time. When the absorbent pad is removed by the user from the chassis, it readily disintegrates and disperses in an excess of water, such as in a toilet bowl.

The use of such a flushable pad is typically restricted to situations without heavy liquid loadings, as may be overnight situations, but rather suit persons—be these male or female—with low to moderate liquid loss of urine and/or menses, who are sufficiently mobile to use toilets, where they may relieve themselves of larger volumes and also can replace a loaded pad.

Thus, in this first aspect, the present invention is a flushable absorbent pad suitable for the second aspect of the present invention, namely a combination of a flushable absorbent pad with an article chassis.

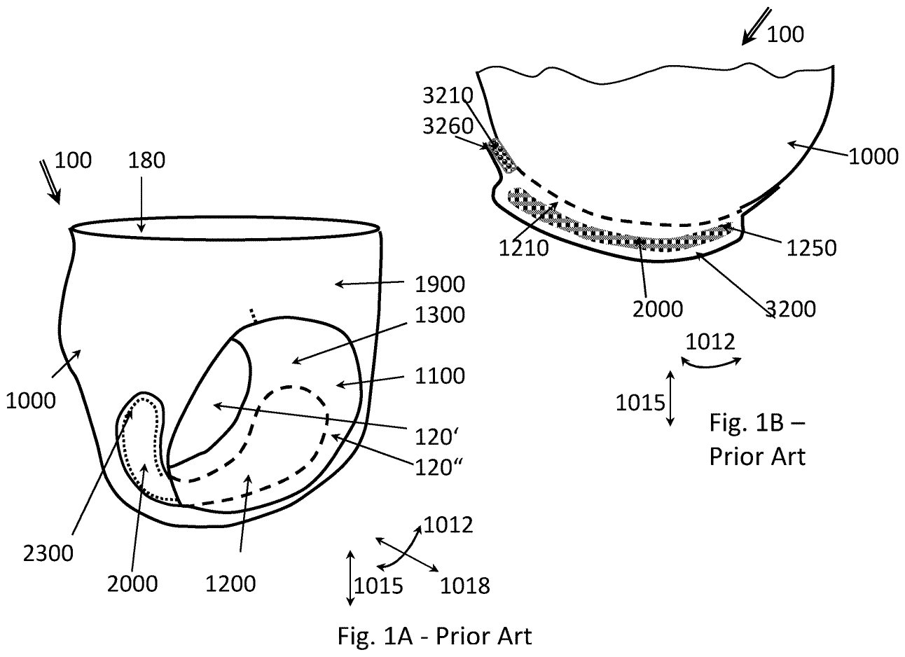

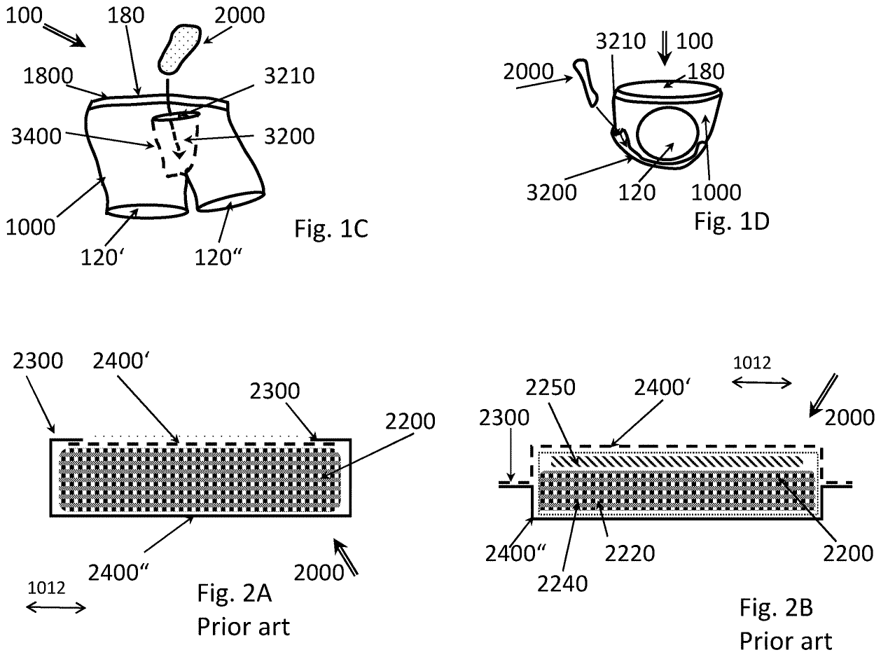

The principles of the present invention are now explained when referring to FIG. 1A to D, and 2A to 2D, which, however, should not be seen limiting to the invention.

As depicted—and generally known in the art—and absorbent pad 2000 can be positioned into a chassis 1000, and the combined system 100 with a waist opening 180 and two leg openings 120 can be worn by the user. In a preferred execution, and as will be explained in more details herein below the chassis 1000 comprises a pocket 3200 with a pocket opening 3210, optionally with a pocket opening closure means 3260, into which the absorbent pad 2000 can be inserted.

When the absorbent pad is loaded, it can be removed by the user, and a fresh pad can be inserted. The article chassis preferably comprises a main region 1300 and an intake zone 1200, the latter to be generally positioned in registry with the pocket 3200 and the absorbent pad 2000 to be positioned therein.

The article chassis 1000 exhibits a width (y−) 1018 and a length (x-) 1012 direction, the first corresponding to a left-right orientation on a user, the second corresponding to a line extending form the front, i.e., navel, through the crotch to the back, i.e., small of the back, of a user. A thickness (z−) direction 1015 extends perpendicularly to both, whereby the terms “upper”/“inner” and “lower”/“outer” correspond to “towards the user” and “away from the user”, respectively. The skilled person will readily apply such terms also to a chassis and an absorbent pad when detached from a user, such as when manufactured or packaged.

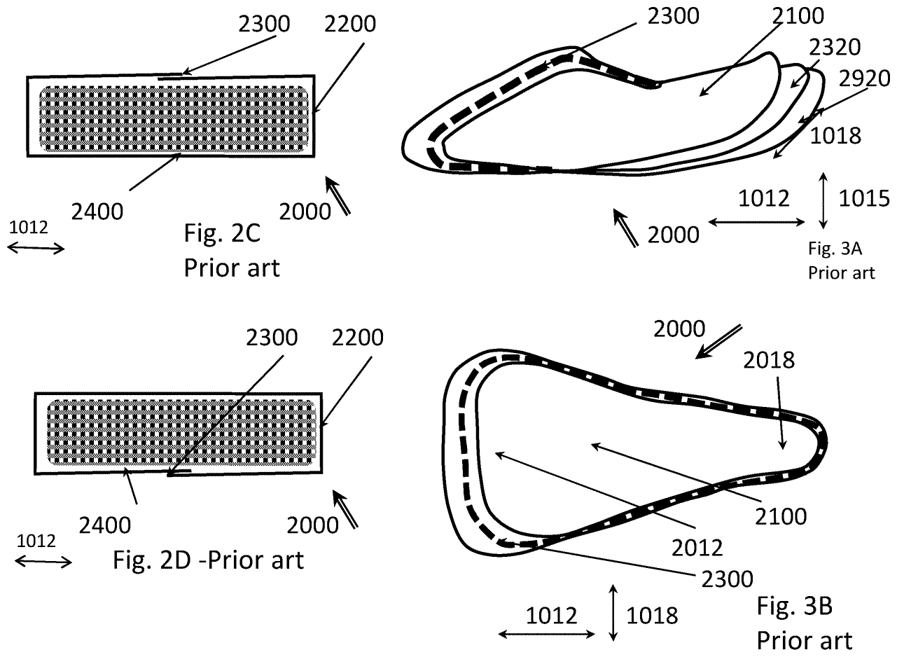

Generally, an absorbent pad for being combined with a chassis as shown in FIG. 2A to D in a cross-sectional view along the cross-directional 1018 center-line 1019 as shown further in FIG. 3A with a partly exploded view and in FIG. 3B in a top view, comprises an absorbent core 2200 and optionally a fluid handling aid 2250, sometimes also referred to as an acquisition-distribution layer between a user-oriented cover 2100, often referred to as topsheet, and an opposite cover 2920.

As shown in FIG. 2A, the absorbent core 2200 may be enveloped by an upper (2400′) and a lower (2400″) wrap material, which are connected to each other by bond lines 2300 along the periphery (FIG. 2B) or along the overlap region of the materials (FIG. 2A). Alternatively, the wrap material may be unitary, and be connected in the overlap region of the “C-fold” (FIGS. 2C and 2D).

The shape of the absorbent pad is not particularly limited, e.g., it may be elongate with the longer extension corresponding to the length direction 1012, thus exhibiting a front part 2012 and a rear part 2018 along the orientation as to be worn on a user, i.e., the front part being forwardly oriented away from the crotch portion of the pad. A particular shape is depicted in FIGS. 3A and 3B as a so called “saddle shape”.

For an absorbent pad according to the present invention to satisfy the requirements of being flushable, readily dispersible and biodegradable, also the materials used therein must satisfy the requirements as such.

Thus, the absorbent core 2000 is made of materials satisfying the flushability criteria according to EDANA GD.4, optionally also the compostability criteria, as described in the TEST SECTION herein below.

Thus, only fully degradable preferably bio-degradable materials are to be used, such as natural based fibrous materials, such as cellulose based pulp, or viscose rayon, or less preferably synthetic materials that are modified to impart sufficient biodegradability, e.g., as described in WO2022/073764 (Gottlieb Binder), referring to materials such as polycaprolactone, polybutyrate or polybutylene succinate, for which the degradation can be accelerated through the use of enzymes.

Further, in order to enhance absorbency properties, particulate material can be added that can absorb water or aqueous solutions at a multiple of its own weight. Such particles—well known as highly absorbent or “superabsorbent polymers”—are currently produced and used at a large scale on a cross-linked polyacrylate basis, however these do presently not satisfy the degradability criteria.

Highly absorbent powders that do satisfy the (bio-)degradability criteria are known in the art, are often though not necessarily based on mildly crosslinked starch, such as from GreenThings GmbH, Germany—FAIRGREEN biologischer Superabsorber, or on modified carboxymethyl cellulose (CMC), such as BioSAP Favor Z4018 from Evonik, Germany. Even though the absorbency and liquid handling properties are still somewhat inferior to the ones of the polyacrylate based ones, these may very well be suited for the present invention, as the absorbent pad can typically be changed more often than a high liquid load pad, such as for overnight loading, would require.

The absorbent capacity of an absorbent pad according to the present invention is primarily given by the one of the absorbent core, this may exhibit an absorbent capacity, as may be measured according to the Centrifuge capacity method as described in the Methods Section herein below, of more than about 10 ml, or more than about 50 ml, or more than about 100 ml and for certain applications also more than about 200 ml, but typically less than about 500 ml or less than about 300 ml. An absorbent pad may further exhibit an area specific absorbent centrifuge capacity of more than about 500 ml/m² or more than about 1000 ml/m², or more than about 2000 ml/m², even more than about 4000 ml/m², and for certain applications more than about 10000 ml/m², but typically less than about 30000 ml/m².

An exemplary absorbent core may be made by intermixing fibers and SAP material within pad forming production line. For example, highly absorbent powders and fibers can be readily combined into an absorbent composite whereby the particles are intermixed with fibers, such as natural fibers like cellulose, which is then compressed, such as with a dot pattern, which leads due to the low amounts of residual moisture of the fibers to hydrogen bonds between the fibers of the composite, thereby also entrapping the particles. Upon wetting, the hydrogen bonds give, and the particles and fibers are released as a first step towards dispersion and subsequent flushability and degradability.

An exemplary absorbent core may be a preformed or “off-line” embossed airlaid material of cellulose fibers with degradable particulate highly absorbent material, whereby the embossing imparts hydrogen bonding between the fibers and thus provides dry integrity but dispersibility upon wetting. Hydrogen bonded air laid materials (HBAL) are generally available such as from McAirlaid's, Germany, under the designation SuperCore SCP-150-TCF, and are also used for the manufacturing of flushable wipes. Another suitable execution is a design following the Gelok™ material commercially available from Gelok Inc., USA, when employing biodegradable superabsorbents, whereby absorbent material is placed between readily disintegrating fluff tissues.

Optionally, the absorbent material in the absorbent core may be further immobilized by immobilization additives, that lose the immobilizing function at the latest when disposed in the toilet. This effect may be achieved by water soluble adhesives, or adhesives that are salt sensitive, such as known from e.g., from EP1280952, to which express reference is made for this effect.

Whilst the immobilization effect may already start during use, this is not considered critical, as long as the material is contained in the absorbent pad, such as by the wrap material, and the absorbent pad in the chassis. Further, the immobilization additive is not to be transferred towards the wearer.

An absorbent core according to the present invention should exhibit a balanced stiffness.

Preferably, the stiffness is such that the application of the absorbent pad with the absorbent core is eased, i.e., exhibiting a dry stiffness according to the Cantilever stiffness test, as described in the TEST Methods herein below of less than about 20°, or less than about 15°, or less than about 10°, or even less than about 5°. However, as will be described herein below, also relatively low stiffness may be compensated by other means, such as insertion aids. During and after use, the stiffness should be low for comfortable wearing and to ease removal. Thus, at a load of 50% of 0.9 w-% saline solution of its design capacity, as may be determined by centrifuge capacity, the absorbent pad should exhibit an overhang length to reach a bending angle of 41.5° of less than about 12 cm, or less than about 10 cm less than about 9 cm, or less than about 8.5 cm.

However, for any such an absorbent core that
may lose particulate material even in a dry state,
may start to disintegrate during use upon liquid load,
and must completely disintegrates in an excess of water,
   it is an important aspect of the present invention that it is enveloped in a wrap material.

Thus, an absorbent pad according to the present invention comprises a wrap material that must satisfy the flushability, dispersibility, and degradability as for the total absorbent pad, but also be liquid permeable at least where it is positioned towards a user.

Suitable materials may be selected from the class of hydroentangled materials, wherein the fibers are entwisted so as to create integrity without additional adhesives. However, the degree of entanglement and the selection of fibers should be selected so as to satisfy the disintegration criteria.

Alternatively, the temporary bonding of the wrap material may also be achieved by selecting materials that allow for hydrogen bonding through compression or crimping, as described for the core, such as well-known from tissues, such as toilet tissues.

Such materials may be delivered as roll stock goods, such as from Suominen, Finland, under the designation Hydraspun® Aquaflow, or Glatfelter, Germany, under the designation DYNAWEB HE G1.5, as made according to the teaching of WO2016/023856, at a basis weight of 55 g/m² or 60 g/m², with target strength values as shown in Table 1.

Such materials are also readily dispersible, by reaching the dispersibility requirement according to the “slosh box test” as per EDANA GD.4 already at less than 30 mins as compared to the minimum requirement of 60 mins.

Another suitable material was sold under the trade designation PRK 20 from Bonded Fiber Fabrics, now Berry Plastics, exhibiting approximate tensile strength values also included in Table 1.

TABLE 1

| | Dynaweb BW | Boded Fiber |
|---|---|---|
| | HE G1.5 | Fabrics |
| Tensile strength MD dry | More than 450 [N/m] | 2950 |
| Tensile strength CD dry | More than 200 [N/m] | 320 |
| Tensile strength MD wet | More than 100 [N/m] | 140 |
| Tensile strength CD wet | More than 65 [N/m] | 7 |

Wrap materials are preferably free of any additional binder, such as glues or adhesives, unless these disintegrate very rapidly. Similarly preferred is that the wrap materials are free of permanent thermofused bonds, such when thermoplastic materials are present and are connected by heat, pressure, ultrasonics or the like, unless the thermoplastic materials are degradable as such.

The wrap materials may be single layer materials, such as a conventional fluff tissue, or may also be composite materials comprising two or more sub-layers, all satisfying the disintegration and biodegradability criteria.

It is an important aspect of the present invention that the absorbent pad maintains sufficient integrity during use, when supported by a fixation aid, such as a chassis pants, but disintegrates quickly after use. Further, absorbent material of the absorbent core needs to be contained, preferably completely contained during manufacturing, transport and use.

Henceforth, an absorbent pad according to the present invention comprises an absorbent core, which is wrapped in a wrap material which if folded around the core so as to completely envelop the core. This enveloping is maintained during use by the combination with the separate fixation aid, preferably chassis pants with a pocket adapted to insert the absorbent pad. The wrapping must be executed such that the material of the absorbent core remains contained therein during use. To this end the wrap material is overfolded along several fold lines around the core.

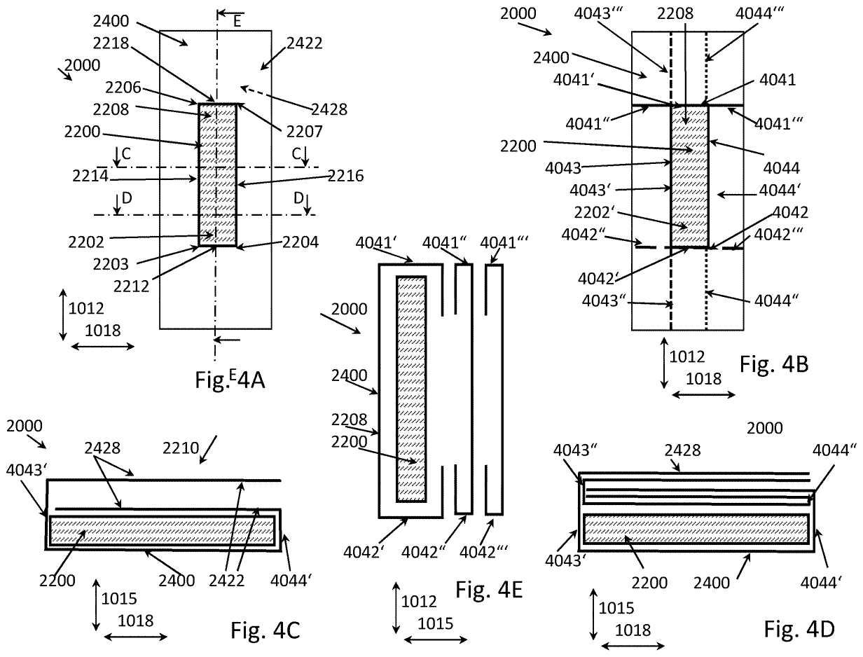

This principle is further explained by referring to FIG. 4A to E. FIG. 4A shows a x− (1012)—y− (1018)—directional top view of a core 2200 and a wrap material 2400, into which the core may be wrapped, prior to any folding. As exemplarily shown in FIG. 4A, both the core and the wrap material the core exhibits exemplarily, but not limiting to the present invention, a rectangular shape, and the core is shown with a front (2202) and a rear (2208) portion.

The wrap material may be a wrap sheet or web 2400, and also the core 2200 may exemplarily be a flat structure with even thickness in the z− (1015) direction, see FIG. 4C. The wrap sheet 2400 may have a width of about three times the width of the core 2200, and a length of up to twice the length of the core, though the exact dimensions may vary as long as the wrap sheet 2400 is sufficiently large to be folded around and cover all surfaces of the core 2200 at least once. The core is now enveloped by folding the wrap sheet 1400 along four fold lines, see FIG. 4B 4041, 4042, 4043, and 4044, each with segments 4041′, . . . adjacent to the core, and segments 4041″, . . . , and 4041′″ extending laterally and longitudinally, respectively, towards the margins of the wrap material, also corresponding to the portions of the edges of the wrap material. Along the fold lines the wrap material is overfolded upwardly over the core, e.g., for the first fold the surface 2422, onto which the core 2200 is placed, is folded towards itself. Whilst the folding can be executed in any order, FIG. 4 depicts exemplarily a preferred order by overfolding
1) rear CD-fold line 4041;
2) front CD fold line 4042;
3) first longitudinal fold line 4043, as may be the left side;
4) second longitudinal fold line 4044, as may be the right side, opposite to the first longitudinal one.

Obviously, folding steps 3) and 4) also overfold the folded portions of the front and rear portions, such that certain regions of the core are covered by multiple layers of the wrap material. FIG. 4C depicts in a width (1018)—thickness (1015) cross-sectional view a portion along the cross-directionally center line CC with lateral folded center portions of the edges 4043′ and 4044′. FIG. 4D depicts a width (1018)-thickness (1015) cross-sectional view in the front portion of the core DD with same fold edges 4043′ and 4044′, but further showing further layers of wrap material as a result of the folding along line 4042, with edges 4043″ and 4044″, respectively. Further FIG. 4E depicts a longitudinal length (1012)—thickness (1015) cross-sectional view EE, with multiple overlying layers in the front and rear portions, as will be discussed in more detail herein below.

It should be noted that the figures are schematic only, and thus do not exactly reflect the impact of the thickness of the wrap material on the exact folding dimensions.

Thus, for such a design it is an important requirement that all four side margins of the core (front 2212, rear 2218, first side 2214, opposite side 2216) are encased by a corresponding fold line, restricting passage of any core material to the outside of the absorbent pad. It is also important to realize, that in such a design not only the sides, but also the corners 2203, 2204, 2206, and 2207 of the absorbent core are well encased.

Whilst the above description focuses on a preferred execution with a unitary wrap material 2400, also multiple wrap sheets may be employed. Referring to FIG. 4F with a view corresponding to the one of FIG. 4A, and FIG. 2G, corresponding to FIG. 4C, two wrap sheets 2400′ and 2400″ are positioned in a partly overlapping arrangement and may be overfolded so as to form two overlapping regions 2210′ and 2210″ in the center region of the absorbent core. The longitudinal end portions of the wrap sheets may then be overfolded as described in the above.

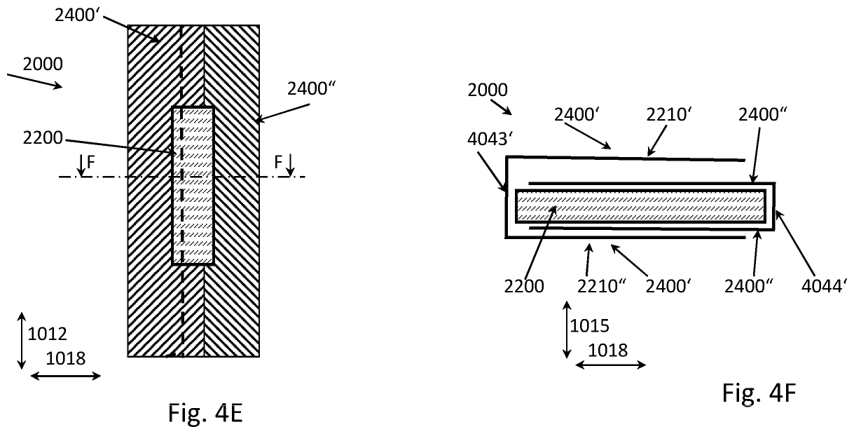

In a preferred execution the design according to the present invention comprises a means for easing the removal of a used pad. A particular design variant is depicted in FIG. 5, showing a core 2200 and a wrap material 2400 as discussed in the above, further comprising a removal aid allowing a user to easily grab the loaded absorbent pad and remove it for disposal. In the exemplary execution as shown in FIG. 5, the removal aid 2500 is formed from an extension of the wrap sheet in the front region 2002 of the absorbent pad 2000. When comparing such an approach to the design as discussed in the context of FIG. 4B, the front folding line 4042 is replaced by two front diagonal fold lines 4045′, 4045″ and optionally a further cross-directionally extending front fold line 4045′″.

However, for certain executions it is not necessary that all four sides of the core need to be encased by fold lines of the wrap material. Referring to FIG. 6, showing a view similar to the one of FIG. 4B and 5, the front fold line 4042 may be omitted, if the wrap material extends in the longitudinal direction sufficiently long beyond the front margin 2212 of the absorbent core 2000, forming a front flap 4046 in the front region of the absorbent pad 2002. The other fold lines 4041, 4043, and 4044 prevent material loss along the other sides, but in the front region the insertion into the pocket of the chassis is sufficient to encapsule the core material. Such a front flap 4046 may exhibit a front opening width 4047 corresponding to the width of the absorbent core and a front flap length 4048 extending longitudinally more than about 10 mm, or more than about 15 mm, or more than about 20 mm beyond the front core margin 2212.

Yet another variant of the design is exemplarily also explained in the context of FIG. 6, wherein the absorbent pad 2000 is adapted to cooperate with an insertion aid 3500 to ease insertion of the absorbent pad into a pocket of a chassis (not shown in FIG. 6).

Such an insertion aid 3500 may be any suitable element exhibiting a flexural rigidity higher than the one of the absorbent pad, which may be combined with the pad during manufacturing of the pad, or by the user upon donning. It may have a various shapes, such as exemplarily shown as a flat tongue blade-shaped insertion aid in FIG. 6. It may also have a curved shape like a shoe-horn, or an uneven thickness e.g., with a bulge at the end which is to be inserted. The insertion aid may satisfy the same flushability and degradability requirements as the absorbent pad, and may thus be discarded after having inserted the absorbent pad into the chassis, or may remain inserted during use. In the latter case, it is preferred that the insertion aid does not reduce the wearing comfort and exhibits a certain suppleness at least in the cross-direction or after it loses its stiffness properties such as by the body heat or liquid. In an alternative, and often preferred, execution, the insertion aid is re-usable and is removed from the absorbent pad once this is positioned properly. This does not present a hygiene issue, as at that point the pad is still unused and clean.

To this end, the absorbent pad comprises an opening for receiving or housing the insertion aid. As depicted in FIG. 6, such an opening may be the front opening through the front flap 4046 as described above.

Whilst the design as depicted in FIG. 4 to 6 represents an execution that allows easy manufacturing due to the rectangular shape of the core and the wrap material and the CD−/MD orientation of the fold lines, for certain executions it may be preferred to use other shapes of the core, which may also create differently shaped absorbent pads after wrapping.

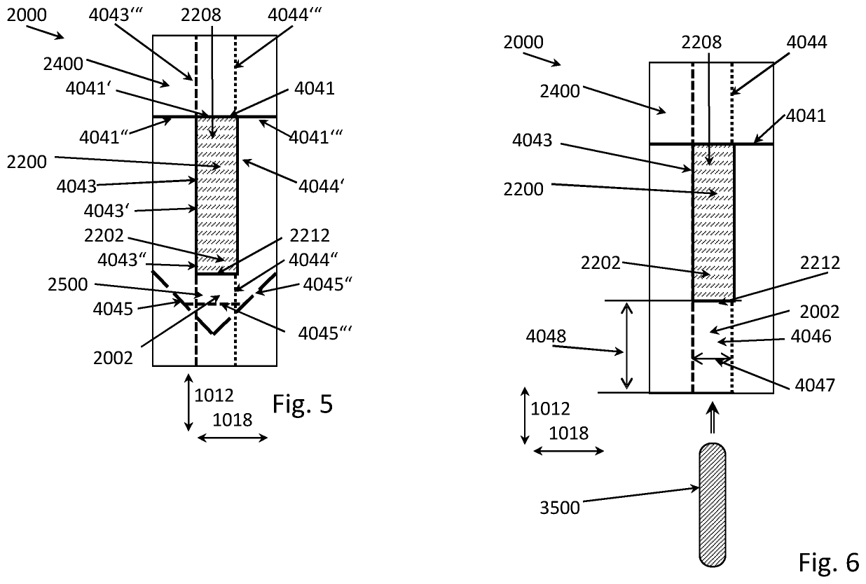

Also for such designs it is important that the migration of core material to the outside is minimized or avoided by enveloping the absorbent core by overfolding the wrap material. Exemplarily, FIGS. 7 A and B depict a rounded absorbent core 2200 in an x (1012)-y (1018)—directional top view (FIG. 7A—in analogy to FIG. 4A) and a y (1018)-z (1015) (width-thickness) directional cross-sectional presentation (FIG. 7B—in analogy to FIG. 4C). Also shown in FIG. 7B is the wrap sheet 2400 overfolded around the core along longitudinal fold lines 4044 and 4043 As in such a design the front, rear and lateral core margins are less defined than in the rectangular design, the enveloping criterion is now formulated via the projection of the core onto a x-y− directionally extending plane 4020, as shown in FIG. 7C, whereby at any point of the contour 4025 of the core 2200 a normal vector 4021 perpendicular to the tangent 4029 in that point and directing away from the core contour 4025 needs to cross the projection of a fold line, e.g., 4033, 4034 in FIG. 7B, of the wrap material onto that plane 4020. Thus, in analogy to FIG. 4B, the rounded core may be enveloped of the four fold lines 4041, 4042, 4043, and 4044, represented by their projections 4031, 4032, 4033, 4034 in FIG. 7C.

However, as exemplarily depicted in FIG. 7D, the fold lines do not need to extend length- and width-wise, but can also be angled thereto, such that a rounded core 2200 may be enveloped by
1) a cross directional fold line 4041;
2) two angled fold lines 4043, 4044;
3) two longitudinal fold lines 4045, 4046.

A further cross-directional fold line 4041 at the front portion of the absorbent pad 2002 can be omitted, if a front opening with a front opening width 4047 is included. In such a case, the criterion that the enveloping with the wrap material should satisfy is that the vectors 4029 directing away from the core do cross a fold line except if they cross the optional front opening line defined by a line as the cross-directional tangent 4029 to the furthermost front point 2201 of the core contour between laterally outermost longitudinally extending tangents to the contour, here shown coinciding with fold lines 4045 and 4046.

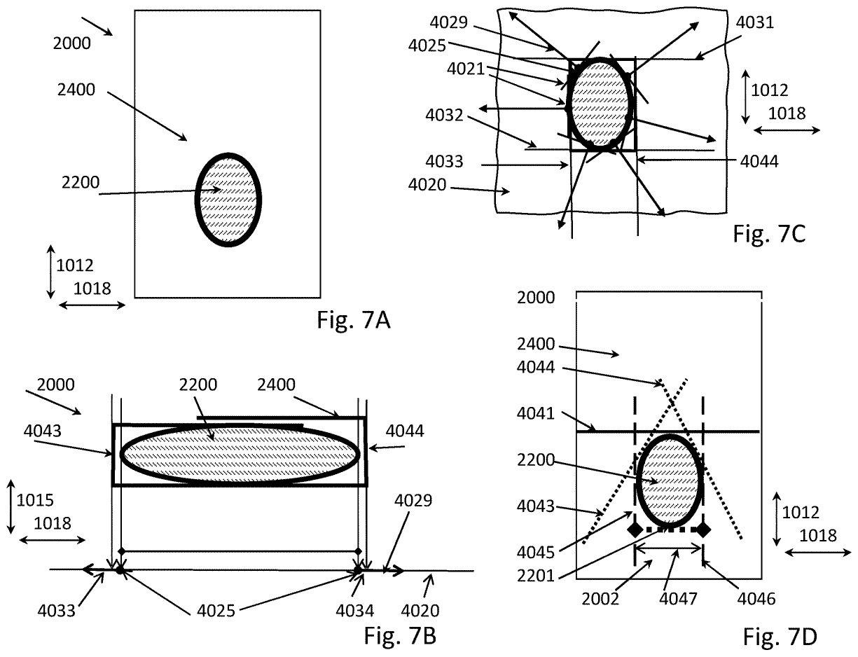

Referring to FIGS. 8A and B, exemplary other core shapes may take the shape of a hexagon or a rounded hourglass, whereby folding may be applied as described for the rectangular or the rounded core. FIGS. 8C and D depict executions where the core is tapering towards the rear portions 2208, whereby the rear cross-directional folding may be omitted when the diagonal folding lines intersect.

In the front. The folding may be omitted, when a design with a front opening is desired, with a front opening width 4047.

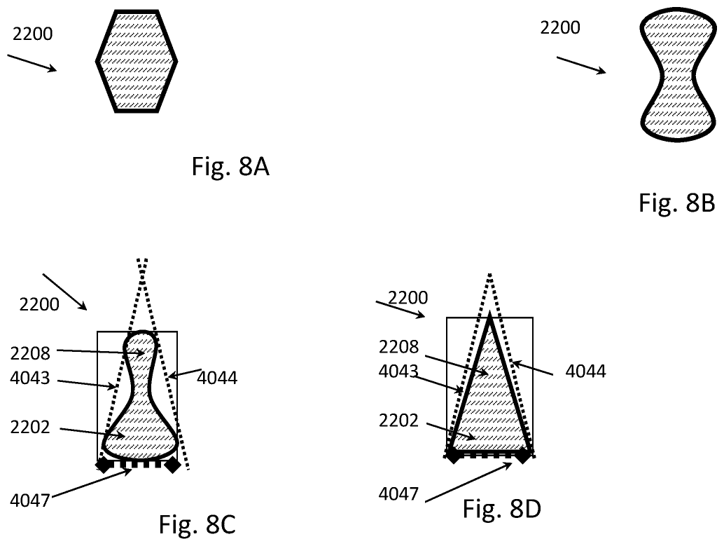

Another suitable design according the present invention is depicted in FIG. 9A in analogy to FIG. 4E with an absorbent core 2200 enveloped in a wrap material 2400 with a “G-type” overfold and longitudinal fold lines 4041 and 4042. In a further processing step, the absorbent pad 2000 is longitudinally overfolded along a width-directional fold line 2040. Such a design provides the option of being combined with an insertion aid 3500, as shown in FIG. 6, which can be readily inserted into the overfolded pad opening 2415.

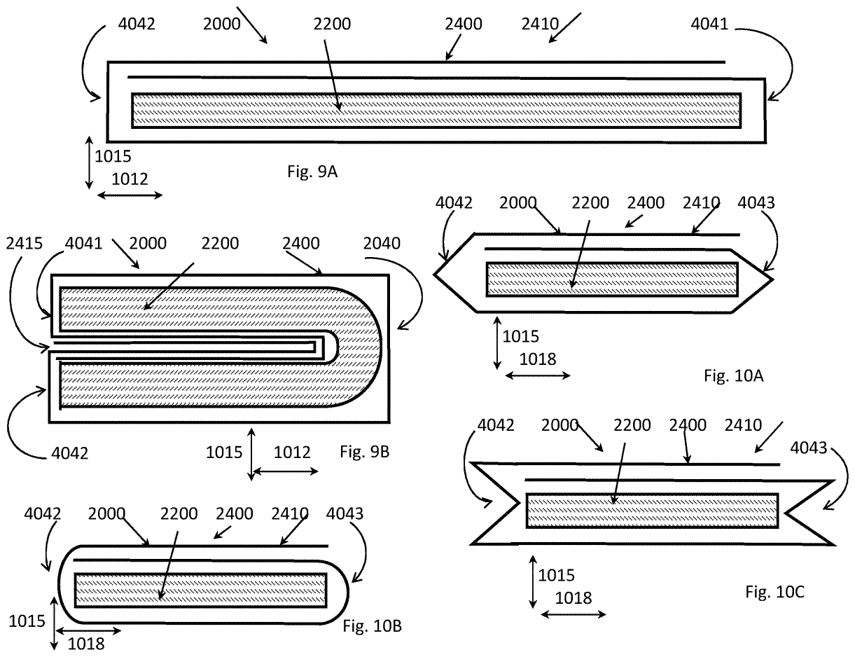

In cross-sectional views, see e.g., FIG. 4 C to D, or 7B, or 9AB, overfolding fold lines were shown as rectangular double folds. The skilled person will readily realize that such shape may either be created by a dual folding step yet being considered as “a” fold line, or it may also be created in a single folding step, see FIG. 10A, shown as a lying “V”. Depending on the distance of the fold line to the core, the thickness of the wrap material and of the absorbent core—both in dry or wetted state—and optionally on any fixation of wrap material or core, the fold line may take the rectangular shape, the “lying V-shape” or even take a rounded shape, as indicated in FIG. 10B. In a further variant in particular for absorbent cores that might increase in thickness upon absorbing fluids, the fold line can be executed as a “lying M” a shown in FIG. 10C, or even in a multifolded “leporello” shape (not shown).

Optionally, the absorbent pad may comprise further additives, especially odour control additives, such as available under the designation ODOgard® Technology from Rem Brands Inc., KY, USA. Whilst such technology may also be applied to washable garments, such as a chassis suitable for the present invention, the odour control effect tends to be reduced through each washing. Applying such additive to the absorbent core delivers a fresh material for each new application of an absorbent pad.

In a particular execution of the present invention, the absorbent pad is free of any additional bonding, i.e., free of glue and adhesives and also no permanent thermoplastic bonding is present.

However, certain bonding of various elements of the absorbent pad may be incorporated, if the above criteria for the enveloping are met and if the bonding opens readily during or after use.

A first of such possible bonding relates to embossing, crimping or pressure bonding, which does not employ any further material but relies on mechanical interaction and or intrinsic bonding aids, such as moisture of e.g., cellulosic materials, as may lead to hydrogen bonding upon compression.

Embossing may be executed in a particular pattern, which may be wetness sensitive, i.e., lose its bonding property upon wetting. In particular, when the absorbent core comprises significant amounts of cellulose, embossing may suitably be employed to immobilize embedded particulates, such as superabsorbent particles, or to increase the bending stiffness of the dry absorbent core for easing insertion. Such embossing may be performed on a “pre-formed” core material or inline on the core just prior to be enveloped or even after the enveloping.

Also, particular bonding additives, such as adhesives or glues, may be employed, also under the proviso that they lose their bonding properties after use at the latest, and that they satisfy the degradability criteria as the other materials of the absorbent pad. Such water sensitive glues are well known in the art, such as available from Intercol BV, The Netherlands, or Beardow & Adams Ltd, UK, or BC Adhesives, WI, USA.

Other suitable additives may be stiffening agents for easing application, that lose their stiffening properties after donning, such as by body temperatures or ion-concentration change, see e.g., EP1280952 (K-C).

However. The application of additional glues or adhesives also carry a disadvantage of processing complication, as drying may be required, but also of inducing a higher digestion burden in any water treatment unit after being flushed though the toilet system.

Of course, the application of such additives may also be combined with embossing, as described above.

Whilst such bonding technologies typically weaken during use due to movement and/or moisturization, it is a particular effect of the present invention that the absorbent pad is adapted to be inserted into a pocket of a chassis, and thus this weakening does not impede the use thereof.

Preferably, the wrapped absorbent pad exhibits a tensile strength along its length direction that is more than twice the tensile strength of the wrap web, measured along the direction corresponding to the length direction of the absorbent pad, preferably more than about 2.5 times, or more than about 3 times, or even more than about 6 times.

The wrapped absorbent pad preferably exhibits a dry stiffness corresponding to the dry stiffness of the absorbent core, as described in the above.

In a loaded state during use, e.g., at a 50% liquid load relative to the centrifuge design capacity of the absorbent pad, the stiffness of the core may be significantly reduced due to the wetness sensitivity of the core materials.

In the further aspect of the present invention, the absorbent core as being fitted into a fixation aid, such as an article chassis, is given support to not disintegrate in spite of the loading and wetting during use. Thus, an absorbent pad is complemented by a chassis that compensates for some in-use deficiencies that the flushable pad would show if used alone.

Referring again to FIGS. 1C and D, the chassis 1000 comprises a pocket 3200 adapted to receive an absorbent pad 2000 according to the first aspect of the present invention in its dry state, where it preferably exhibits a stiffness or rigidity to allow easy insertion or is adapted to be inserted by an insertion aid. The pocket of such a chassis containing an absorbent pad according to the present invention will provide sufficient protection against mechanical stresses as induced under normal wearing conditions, and hence ensures that the absorbent pad will not lose liquid handling performance due to strong deformation and especially due to premature disintegration of the absorbent core. In a particular execution, the pocket can expand so as to allow for swelling of the absorbent pad during use. Such an expansion can be achieved by using elastic or at least extensible material, wherein the first refer to materials that comprise elastomeric materials and thus exhibit higher retractive forces than the latter, as may be made from essentially in-elastic materials, such as cotton yarns, that are manufactured into a web, such as by knitting, the is flexible and extensible with moderate retraction forces, such as known from e.g., cotton underwear.

Further, the chassis of a system according to the present invention may provide liquid sealing functionality, especially if the absorbent pad does not comprise a built-in liquid barrier layer on the its opposite side. Preferably, the chassis may comprise further liquid barrier properties, such as wicking barriers for preventing lateral liquid distribution.

The stiffness of the chassis is less relevant but should satisfy normal wearing comfort requirements and not hinder insertion of the absorbent pad. A further highly preferred feature of the chassis is that it should not overly occlude the skin, but rather allow aeration and even wetness removal such as by being breathable, preferably by comprising material exhibiting a moisture vapour transmission rate of more than about 2000, or more than about 7500, or more than about 10000, or even more than about 12000, all expressed in g/m²/24 hrs.

It should be noted that the system of the chassis as fixation aid and the absorbent pad does not need to satisfy the flushability criteria, e.g., when the slosh box test is executed with an absorbent pad inserted in a pocket of the chassis, the absorbent pad does not need to disintegrate.

Preferably, the chassis comprises breathable fabric or textile webs. Such chassis—pad systems are generally well known in the art, see above referenced U.S. Pat. No. 8,192,415(P&G) for disposable chassis, or more preferably for a washable and reusable chassis, as described in more detail in co-pending application WO2023/180584 (Pelz), to which express reference is made for the design and construction of a chassis for receiving an absorbent pad.

### Test Methods

### EDANA Flushability Requirements GD.4

EDANA, Belgium, has established flushability criteria especially for nonwoven materials like wipes, comprising the criteria of
FG501.R1(18): Toilet and Drain-line Clearance Test;
FG502.R1(18): Slosh Box Disintegration Test;
FG503.R1(18): Household Pump Test;
FG504.R1(18): Settling Test;
FG505.R1(18): Aerobic (A) Biodisintegration/(B) Biodegradation Tests;
FG506.R1(18): Anaerobic (A) Biodisintegration/(B) Biodegradation Tests;
FG507.R1(18): Municipal Sewage Pump Test;

The skilled person will readily realize that articles—such as absorbent pads—should satisfy the same criteria in tests just adapted to cope with potentially different shape of the article. Similarly, also the tests may be applied to materials as used for the construction of such articles, also adapted to reflect the different shape—for example, particulate or loose fibrous material will automatically satisfy the Slosh box Disintegration Test.

### Composting Test Methods

DIN EN 13432: 2000-12: Requirements for packaging recoverable through composting and biodegradation—Test scheme and evaluation criteria for the final acceptance of packaging;
DIN EN 14995: Plastics—Evaluation of compostability—Test scheme and specifications;
ISO 18606:2013-01 Packaging and the environment—Organic recycling;
NF T51-800:2015: Home or garden composting;
ASTM D 6868“Standard Specification for Labeling of End Items that Incorporate Plastics and Polymers as Coatings or Additives with Paper and Other Substrates Designed to be Aerobically Composted in Municipal or Industrial Facilities”;
ASTM D 6400:2023“Standard Specification for Labeling of Plastics Designed to be Aerobically Composted in Municipal or Industrial Facilities”;

### Degradability—Chemical Oxygen Demand

DIN/DEV 38409: 2011-09 German standard methods for the examination of water, waste water and sludge; General information (group A) Part 1: Recording of analysis results (A 1);
ÖNORM M 6265: 1091 Water analysis—Determination of the chemical oxygen demand;
ASTM D 1252:2006“Standard Test Methods for Chemical Oxygen Demand (Dichromate Oxygen Demand) of Water”;

### Degradability—Biological Oxygen Demand

DIN EN ISO 5815-1:2020-11 Water quality—Determination of biochemical oxygen demand after n days (BODₙ)—Part 1: Dilution and seeding method with allylthiourea addition
DIN 38 409—H51: German standard methods for the examination of water, waste water and sludge; general measures of effects and substances (group H); determination of the biochemical oxygen demand after n days, dilution method (dilution BSBₙ)
DIN 38 409—H52: German standard methods for the examination of water, waste water and sludge; parameters characterizing effects and substances (group H); determination of oxygen depletion in after n days.

Tensile strength according to EDANA NWSP 110.4.R0 (15), at 25 mm strip width and constant rate of extension.

Cantilever Stiffness Test EDANA NWSP 090.1 (R0 (15)

At the dry state, the angle for an overhang length of 160 mm is recorded.

For the wet state, the material or article was loaded with 0.9% saline solution up to 50% of its design capacity, as may be determined by the centrifuge capacity absorption, and the overhanging length to reach an angle of 41.5° is recorded

Absorbent Capacity—Centrifuge capacity EDANA NWSP 241.0 R0 (15), as may be adapted such that the particulate material is replaced by a web material or composite. The result is the weight specific absorbency, expressed in grams liquid per gram of material. The Design Capacity of an article reflects the overall capacity by multiplying the specific capacity by the amount of respective material.

Moisture vapour Transmission Rate according to ASTM E96-80, expressed in g/m²/24 hrs.

1-7. (canceled)

8. A flushable and degradable absorbent pad comprising an absorbent core and a wrap material,
exhibiting a length (x-), width (y−), and thickness (z−) direction and comprising a front, a rear, and two lateral portions,
wherein
said absorbent core comprises absorbent material, preferably comprising fibrous and particulate material,
and said wrap material is overfolded along fold lines,
and covers all x-y− directionally extending surfaces of said absorbent core by at least one layer thereof,
and wherein further said fold lines encircle said absorbent core at least laterally and rearwardly,
and optionally leave a front opening section un-overfolded,
wherein further,
when the contour of said absorbent core and said fold lines are projected onto an x-y− directional plane,
all vector lines directing away at 90° from said contour of said absorbent core
are crossing a projection of a fold line,
except if they cross a projection of said front opening section, if present, which is positioned width-wise extending tangentially at the furthermost front point of the core contour
between laterally outermost longitudinally extending tangents to the contour.

9. An absorbent pad according to claim 8, further satisfying one or more of the elements selected from the group consisting of
a) said absorbent pad being essentially free of adhesive or thermoplastic bonding;
b) said absorbent pad being essentially unbonded or by mechanically induced hydrogen bonding only;
c) said absorbent pad exhibiting an absorbent centrifuge capacity of
c1) more than about 500 ml/m², preferably more than about 1000 ml/m², more preferably more than about 2000 ml/m², and most preferably of more than about 3000 ml/m²; or
c2) less than 30000 ml/m². preferably less than about 20000 ml/m²;
d) said absorbent pad comprises absorbent particles based on modified starch;
e) said absorbent pad comprises cellulose based fibers;
f) said absorbent core comprises wetness sensitive embossing pattern.

10. An absorbent pad according to claim 8, further comprising one or more of the elements selected from the group consisting of
g) said wrapping is essentially unbonded or by mechanically bonding induced hydrogen bonding and/or hydroentangling only;
h) said wrapping being essentially free of adhesive or thermoplastic bonding;
i) said wrapping comprises overfold lines of said wrap web that are completely circumscribing said absorbent core;
j) said wrap web comprises a wetness sensitive embossing pattern.

11. An absorbent system comprising an absorbent pad according to claim 8, further comprising
a re-usable chassis comprising a pocket adapted to receiving said pad.

12. An absorbent system according to claim 11,
wherein said absorbent pad does not disintegrate upon submitting the system with an absorbent pad inserted into said pocket to the slosh box test as modified to apply to an article.

13. An absorbent system according to claim 11, wherein
said absorbent core
is exhibiting along the length direction of the absorbent pad
a dry stiffness of less than 20°, preferably less than 15°, more preferably less than about 10°, according to the Cantilever test, as described herein,
and
and a wet stiffness at 50% of its design capacity, as defined herein of less than about 12 cm, preferably less than about 10 cm, more preferably less than about 9 cm, each to reach a bending angle of 41.5° according to the Cantilever test, as described herein;

[symbol]

14. An absorbent system according to claim 11,
wherein said absorbent core is consisting essentially of materials satisfying EDANA GD.4 flushability test requirements, adapted to the materials as described herein.,
and wherein said absorbent core satisfies
k) flushability criteria as described herein;
l) industrial composting according to DIN EN 13432;
m) garden composting according to NF T51-800:2015.;
n disintegrating faster than said wrap web in the slosh box test included in said GD.4 requirements.
